(19) United States
(12) Patent Application Publication
(10) Pub. No.: US 2007/0207331 A1
(43) Pub. Date: Sep. 6, 2007

(54) Azo compounds and coating compositions containing the azo compounds

(76) Inventors: Jason Clay Pearson, Kingsport, TN (US); Max Allen Weaver, Kingsport, TN (US); Jean Carroll Fleischer, Kingsport, TN (US); Greg Alan King, Mount Carmel, TN (US)

Correspondence Address: Brett L. Nelson, Eastman Chemical Company, P.O. Box 511, Kingsport, TN, 37662-5075, US

(21) Appl. No.: 11/368,580

(22) Filed: Mar. 6, 2006

(51) Int. Cl. B32B 27/00; C08K 5/23; C07D 409/02; C07D 401/02

(52) U.S. Cl. 428/500; 524/715; 534/766; 534/771

(57) ABSTRACT

Disclosed are azo colorant compounds containing one or more ethylenically-unsaturated groups polymerizable or copolymerizable with ethylenically-unsaturated compositions typically used in coating compositions. The azo colorantcompounds are polymerizable by photopolymerization or free radical polymerization techniques with ethylenically-unsaturated monomers. The copolymerized colorants produce colored compositions such as colored acrylic polymers, e.g., polymers produced from acrylate and methacrylate esters, colored polystyrenes, and similar colored polymeric materials derived from other ethylenically-unsaturated monomers. The ethylenically-unsaturated colorant compounds may be used in coatings that are applied to wood, glass, metal, thermoplastics and the like.

### FIELD OF THE INVENTION

This invention pertains to novel, azo colorant, or dye, compounds containing one or more ethylenically-unsaturated (vinyl) groups that are polymerizable with ethylenically-unsaturated monomers. When copolymerized with one or more other ethylenically-unsaturated monomers, the azo colorant compounds produce colored compositions polymeric compositions. The present invention also pertains to processes for preparing the polymerizable azo colorant compounds, to coating compositions containing one or more of the ethylenically unsaturated azo colorant compounds and to coating produced from such coating compositions.

### BACKGROUND OF THE INVENTION

Colorant compounds containing ethylenically-unsaturated groups are known. For example, U.S. Pat. No. 5,188,641 discloses a colored polymer containing repeat units derived from at least one azo dye which is free from water-solubilizing groups and contains at least one polymerizable olefinically unsaturated group and has the formula:
A-N═N-E

wherein A represents the residue of a carbocyclic or heterocyclic diazotisable amine containing at least one electron-withdrawing substituent and E represents the residue of a carbocyclic or heterocyclic coupling component with the proviso that when both A and E are carbocyclic, the residue of the diazotisable amine does not contain a nitro or chloro substituent except when another different electron-withdrawing substituent is also present, the or each polymerizable group being attached to a carbon atom forming part of residue A or E. The polymerizable azo colorant compounds disclosed in U.S. Pat. No. 5,188,641 include compounds wherein A is the residue of certain carbocyclic diazotizable amines and E is the residue of a pyridone compound bearing an ethylenically-unsaturated group, i.e., either 1-[2,3-bis(acryloyl-oxy)prop-1-yl]-3-cyano-2-hydroxy-4-methylpyrid-6-one (Coupler 1) or 1-(3-acryloyloxy-n-propyl)-3-cyano-2-hydroxy-4-methylpyrid-6-one (Coupler 2). The following azo compounds of formula A-N═N-E are disclosed in examples of U.S. Pat. No. 5,188,641:

| Example | A | E |
|---|---|---|
| 88 | 2-Chloro-4-Nitrophenyl | Coupler 1 |
| 103 | 4-Nitrophenyl | Coupler 1 |
| 107 | 4-Vinylphenyl | Coupler 2 |
| 108 | 4-Acryloyloxyethylphenyl | Coupler 2 |

It has been found that some of these prior art compounds cannot be prepared in satisfactory yields and/or purities when using conventional synthesis procedures. A typical procedure comprises the steps of (1) diazotizing a carbocyclic aryl amine, (2) coupling the resulting diazonium salt with a pyridone coupling component to form an intermediate azo compound wherein the coupling component does not contain an ethylenically-unsaturated group and (3) reacting the intermediate azo compound with a compound that provides the coupling component with an ethylenically-unsaturated group. When the intermediate azo compound is reacted with a compound that provides the coupling component with an ethylenically-unsaturated group in the presence of an organic solvent, insoluble material forms in pieces or chunks.

### BRIEF SUMMARY OF THE INVENTION

The azo compounds provided by the present invention have formula I: 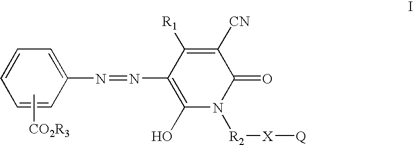
wherein:

R₁ is selected from C₁-C₆ alkyl and aryl;

R₂ is selected from C₂-C₁₀-alkylene, —CH₂-cyclohexylene-CH₂— and —(C₂-C₄-alkylene-Y)ₙ-C₂-C₄-alkylene;

R₃ is selected from C₃-C₁₂-alkyl, C₃-C₈-cycloalkyl and —(C₂-C₄-alkylene-Y)ₙ-R₄;

R₄ is selected from hydrogen, C₁-C₆-alkyl, substituted-C₁-C₆-alkyl, C₃-C₈-cycloalkyl and aryl;

X is selected from —O—, —NH—, —N(SO₂R₅)— and —N(R₅)—;

R₅ is selected from C₁-C₆-alkyl, C₃-C₈-cycloalkyl and aryl;

Y is selected from —O—, —S—, —N(R₅)—, —N(COR₅)— and —N(SO₂R₅)—;

n is 1 to 3; and

Q is an ethylenically unsaturated, polymerizable group selected from groups having structures 1-10: 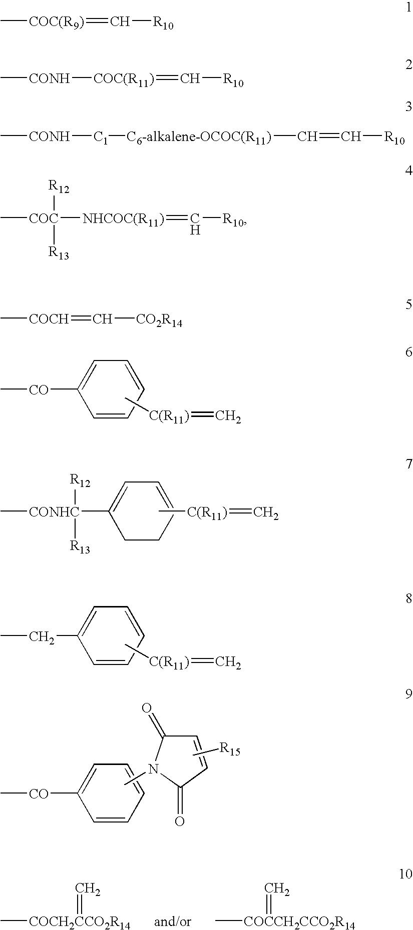
wherein:

R₉ is C₁-C₆-alkyl;

R₁₀ is hydrogen; C₁-C₆-alkyl; phenyl; phenyl substituted with one or more groups selected from the group consisting of C₁-C₆-alkyl, C₁-C₆-alkoxy, —N(C₁-C₆-alkyl), nitro, cyano, C₁-C₆-alkoxycarbonyl, C₁-C₆-alkanoyloxy and halogen; 1- or 2-naphthyl; 1- or 2-naphthyl substituted with C₁-C₆-alkyl or C₁-C₆-alkoxy; 2- or 3-thienyl; 2- or 3-thienyl substituted with C₁-C₆-alkyl or halogen; 2- or 3-furyl; or 2- or 3-furyl substituted with C₁-C₆-alkyl;

R₁₁ is hydrogen or C₁-C₆-alkyl;

R₁₂ and R₁₃ are, independently, hydrogen, C₁-C₆-alkyl, or aryl; or R₁₂ and R₁₃ may be combined to represent a —[—CH₂—]₃₋₅— radical;

R₁₄ is hydrogen, C₁-C₆-alkyl, C₃-C₈-alkenyl, C₃-C₈-cycloalkyl or aryl; and

R₁₅ is hydrogen, C₁-C₆-alkyl or aryl.

The azo compounds of formula I are economical and may be polymerized, e.g., by photopolymerization or free radical polymerization, with various ethylenically-unsaturated monomers to produce yellow coatings. The azo compounds exhibit improved solubility in solvents and/or polymerizable, ethylenically-unsatruated monomers and improved storage stability relative to structurally similar azo compounds known in the art. The improvements in solubility are attributed to the alkyl groups that are selected for the carboxyl substituent on the aniline coupling component. The improvements in storage stability are attributed to the lower reactivity of the ethylenically unsaturated substituent on the pyridone coupling component.

A second embodiment of the present invention is a coating composition comprising (i) one or more azo compounds of formula I; (ii) one or more ethylenically-unsaturated monomers; and (iii) a photoinitiator. Another embodiment of our invention is a coating resulting from the copolymerization of at least one azo compound of formula I with one or more ethylenically-unsaturated monomers.

### DETAILED DESCRIPTION

In the above description of the azo compounds of formula I, the term “C₁-C₆-alkyl” is used herein to denote a straight or branched chain, saturated aliphatic hydrocarbon radical containing one to six carbon atoms. The term “substituted-C₁-C₆-alkyl” is used herein to denote a straight or branched chain, saturated aliphatic hydrocarbon radical containing one to six carbon atoms and these radicals optionally substituted with one to three groups selected from hydroxy, halogen, cyano, aryl, aryloxy, arylthio, C₁-C₆-alkylthio, C₁-C₆-alkylsulfonyl, C₃-C₈-cycloalklyl, C₁-C₆-alkanoyloxy, C₁-C₆-alkoxycarbonyl and —(O—C₂-C₄-alkylene)ₙR₆; wherein R₆ is selected from hydrogen, C₁-C₆-alkoxy, halogen, hydroxy, cyano, C₁-C₆-alkanoyloxy, C₁-C₆-alkoxycarbonyl, aryl, and C₃-C8-cycloalkyl; n is as previously defined.

The term “C₃-C₁₂-alkyl” is used herein to denote a straight or branched chain, saturated aliphatic hydrocarbon radical containing three to twelve carbon atoms and these radicals optionally substituted with one to three groups selected from hydroxy, C₁-C₆-alkoxy, halogen, cyano, aryl, aryloxy, arylthio, C₁-C₆-alkylthio, C₁-C₆-alkylsulfonyl, C₃-C₈-cycloalklyl, C₁-C₆-alkanoyloxy, C₁-C₆-alkoxycarbonyl and —(O—C₂-C₄-alkylene)ₙR₆, wherein R₆ and n are as previously defined. Branched hydrocarbon radicals are preferred. The terms “C₁-C₆-alkylthio”, “C₁-C₆-alkylsulfonyl”, “C₁-C₆-alkoxy”, “C₁-C₆-alkoxycarbonyl” and “C₁-C₆-alkanoyloxy” are used herein to denote the radicals —SR₇, —SO₂R₇, —OR₇, —CO₂R₇, and —OCOR₇, respectively, wherein R₇ is a C₁-C₆-alkyl radical as previously defined.

The terms “C₂-C₄-alkylene” and “C₂-C₁₀-alkylene” are used to denote divalent, straight- or branched-chain, hydrocarbon radicals, containing two to four and two to ten carbons atoms, respectively, and these radicals substituted with one or two groups selected from C₁-C₆-alkoxy, C₁-C₆-alkanoyloxy, hydroxy, aryl and halogen. The term “C₃-C₈-cycloalkyl” is used to denote a saturated, carbocyclic, hydrocarbon radical having three to eight carbon atoms, optionally substituted with at least one C₁-C₆-alkyl.

The terms “aryl”, “aryloxy” and “arylthio” as used herein denote the radicals —R₈, —O—R₈ and —S—R₈ wherein R₈is selected from phenyl and naphthyl and these radicals containing one to three groups selected from C₁-C₆-alkyl, substituted C₁-C₆-alkyl, C₁-C₆-alkylthio, C₁-C₆-alkylsulfonyl, C₁-C₆-alkanoyloxy, C₁-C₆-alkoxycarbonyl, hydroxy, cyano, trifluoromethyl and halogen. The term “arylene” is used herein denotes 1,2-, 1,3-, and 1,4-phenylene radicals and these substituted with at least one group selected from C₁-C₆-alkyl, C₁-C₆-alkoxy and halogen. The term “halogen” is used to denote bromine, chlorine, fluorine and iodine.

The polymerizable yellow azo colorants of formula I may be prepared by one of the following two methods: 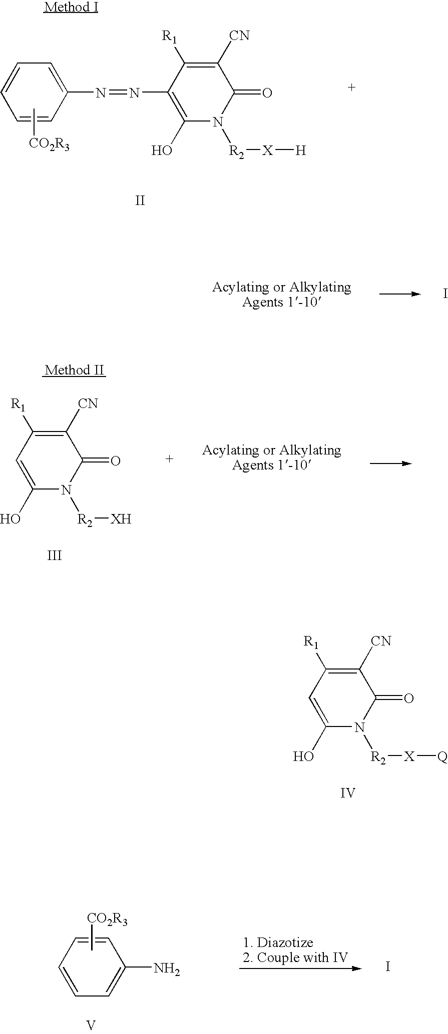
In Method I, intermediate compounds of formula II are reacted with the ethylenically unsaturated alkylating or acylating agents 1′-10′ to give I. In Method II, azo coupling components III are reacted with the ethylenically unsaturated alkylating or acylating agents 1′-10′ and then the intermediate coupling components IV are coupled with the diazonium salt of V prepared by conventional diazotization and coupling procedures. Intermediate compounds of Formula II are prepared by diazotizing aromatic amines of Formula V by conventional diazotization procedures and coupling with couplers of Formula III.

Typical diazotizable aminobenzoic acid esters of Formula V include but are not limited to isopropyl, iso-butyl, sec-butyl, tert-butyl, n-butyl, n-amyl, tert-amyl, isoamyl, n-hexyl, 2-ethylbutyl, 2-methylpentyl, 2-ethylhexyl, 3,7-dimethyloctyl, 1-ethyl-1,5-dimethylhexyl, 2-ethoxyethyl, 2-(2-ethoxyethoxy)ethyl, 2-isopropoxyethyl, 2-(2-isopropoxyethoxy)ethyl, cyclopentyl and cyclohexyl esters of anthranilic acid. Preferred compounds of Formula V are the anthranilate esters wherein R₃ is selected from a branched alkyl groups containing from four to about eight carbon atoms and —CH₂CH₂(OCH₂CH₂)₁₋₃O—R₄, wherein R₄ is C₁-C₆-alkyl.

The colorants of formula I may be prepared by reacting intermediate azo compounds of formula II with the acylating or alkylating agents having formulas 1′-10′ containing ethylenically unsaturated groups: 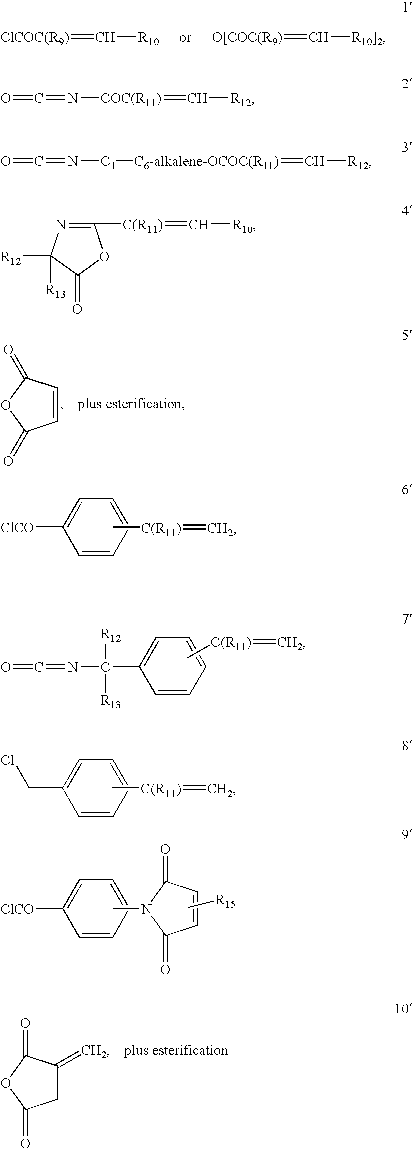
wherein R₉, R₁₀, R₁₁, R₁₂, R₁₃, R₁₄ and R₁₅ are as defined previously. Alternatively, the acylating or alkylating agents of formulas 1′-10′ may be reacted with intermediates III to prepare coupling component IV.

The intermediate coupling components of formula III are prepared according to the following procedures: 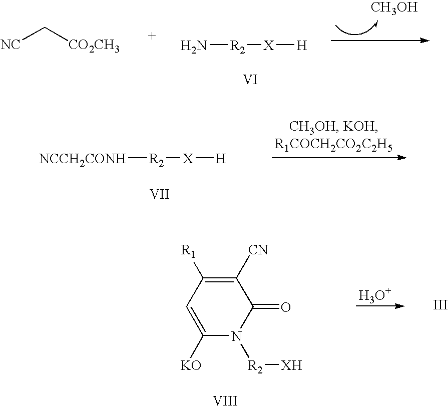
wherein a cyanoacetic ester, preferably the methyl ester, is treated with an amine (VI) and undergoes ester-amide interchange to provide the substituted cyanoacetamide (VII), which is treated with an alkyl aroyl or C₁-C₆-alkanoylacetate, preferably ethyl acetoacetate, in the presence of base, for example an alkali metal hydroxide, in an alcohol, such as C₁-C₄-alcohols, to provide the N-substituted-3-cyano-6-hydroxy-4-C₁-C₆alkyl/aryl-2-pyridone salt (VIII), which upon acidification provides the intermediate azo coupling component III (see J. Org. Chem., 1960, 25, 560). If desired, the salts of the pyridone coupler (VIII) may be used in the azo coupling reaction.

### EXAMPLES

The preparation of the polymerizable yellow azo compounds of formula I is further illustrated by the following examples wherein all percentages are by weight unless specified otherwise.

### Example 1

Methyl cyanoacetate (198 g, 2.0 moles) was stirred and heated to about 100° C. and 2-aminoethanol (122 g, 2.0 moles) was added dropwise over 2.0 hours at about 95-105° C. while methanol was collected in a Dean-Stark trap. The reaction mixture was heated to about 150° C. and held at that temperature until methanol distillation stopped. The reaction mixture was allowed to cool to room temperature. Ethyl acetoacetate (260 g, 2.0 moles) was added and the reaction mixture was heated to reflux. A solution of potassium hydroxide (125 g) dissolved in methanol (300 mL) was added dropwise and refluxing was continued for about 7.0 hours. After 2-3 hours of heating, a rather thick slurry of the potassium salt of the pyridone product resulted. The reaction mixture was allowed to cool and the white solid was collected by filtration, washed with a small amount of cold methanol and dried in air to give 314 g of product. The potassium salt was dissolved in water (500 mL) and stirred while being carefully acidified with concentrated hydrochloric acid (100 mL). A white precipitate formed and was collected by suction filtration, washed with cold water and air dried to give 110 g of 3-cyano-6-hydroxy-1-(2′-hydroxyethyl)-4-methyl-2-pyridone (28% of theory).

Sulfuric acid (82.8 mL, 1.49 moles) was added to a clean, dry 1 L flask equipped with a thermometer and a mechanical stirrer. The reaction vessel was lowered into an ice water bath until the temperature was reduced to about 10° C. To the reaction vessel was added about 12.35 g (0.18 moles) of sodium nitrite in several small quantities at a rate such that the temperature did not exceed 70° C. The reaction mixture was allowed to cool to 0° C. and 172 mL of 1:5 acid (1 part propionic acid; 5 parts acetic acid by weight) were added over 45 minutes at such a rate to keep the temperature below 3° C. To the reaction solution at 0° C. was added 2-ethylhexyl anthranilate (45.0 g, 0.18 moles) over 30 minutes at such a rate to maintain the temperature below 6° C. Additional 1:5 acid (150 mL) was added over 10 minutes at such a rate to keep the temperature below 3° C. The resulting solution was stirred for 2 hours between 0 and 5° C. The reaction solution containing the diazonium salt was added in small portions along with 250 g of ice such that the reaction temperature did not exceed 5° C. to a large 12 L reaction vessel equipped with a mechanical stirrer and containing an aqueous solution of 3-cyano-6-hydroxy-1-(2′-hydroxyethyl)-4-methyl-2-pyridone potassium salt (39.95 g, 0.172 moles), 27.24 g of a 50% solution of sodium hydroxide (0.34 moles), 1000 g ice and 1000 g water. The reaction mixture was allowed to warm to ambient temperature while being stirred for 1 hour, then an additional 1400 g of water was added. The reaction mixture was stirred for an additional 30 minutes then heated to 70° C. using steam. The reaction mixture was allowed to cool to about 45° C. and the solid intermediate azo compound was collected by suction filtration. The filter cake was washed with 1 L of hot water and then allowed to dry on the filter overnight to give 123.18 g of wet press cake of intermediate azo compound (theory: 78.18 g).

The press cake of the above intermediate azo compound was added to a 2 L round bottomed flask equipped with a mechanical stirrer, Dean-Stark trap and heating mantle. Toluene (700 mL) was added. The mixture was stirred and refluxed until distillation stopped. Approximately 48 mL water were removed. Additional toluene (200 mL) was removed by distillation. The mixture was allowed to cool to 80° C. and dibutyltin dilaurate (1.6 mL) was added followed by 3-isopropenyl-α,α-dimethylbenzyl isocyanate (36.36 g, 0.181 moles). The reaction progress was monitored using silica gel thin layer chromatography (TLC) (33% ethyl acetate in heptane). Complete conversion was achieved after 1 hour. The reaction vessel was allowed to cool to 40° C. and added over about 30 minutes to a stirred 4 L beaker containing 2 L of heptane. A free flowing yellow solid formed. The mixture was stirred for 30 minutes and the yellow solid was collected by suction filtration. The cake was washed twice with 250 mL of heptane and allowed to dry on the filter overnight. The filter cake was placed into a vacuum oven at 40° C. with a slight ingress of dry nitrogen for 48 hours to give 112.8 g of dry azo compound product cake (90% yield). Field desorption mass spectrometry (FD-MS) was used to confirm the azo compound product has the structure below. When dissolved in N,N-dimethylformamide (DMF) an absorption maximum at 430 nm was observed in the UV-Visible absorption spectrum.

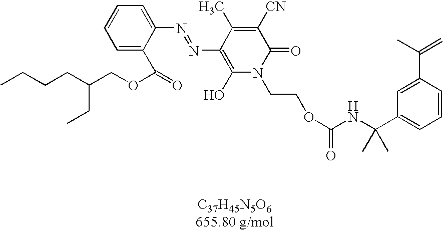

### Example 2

Nitrosylsulfuric acid (40%, 6.4 g, approximately 0.02 mole) was added to a 100 mL round bottomed flask equipped with a magnetic stir bar and ice water bath. To the stirred solution was added 1:5 acid (20 mL) was added in small portions such that the temperature did not exceed 25° C. The reaction solution was further cooled and isobutyl anthranilate (3.86 g, 0.02 mole) was added at about 0-5° C. with stirring and cooling, followed by an additional quantity (20 mL) of 1:5 acid. The diazotization reaction solution was stirred at 0-5° C. for about 2 hours then added dropwise at less than about 5° C. to a solution of 3-cyano-6-hydroxy-1-(2′-hydroxyethyl)-4-methyl-2-pyridone potassium salt (4.64 g, 0.02 mole), dissolved into an ice water mixture (100 mL). The coupling mixture was allowed to sit in the ice water bath with occasional stirring about 45 minutes and diluted with water (100 mL). The yellow, intermediate azo compound was collected by vacuum filtration, washed hot water and air dried to give 6.31 g of yellow azo product (79% of the theoretical yield). The intermediate azo product has the following structure (confirmed by FDMS):

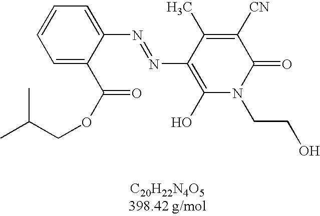

The intermediate azo compound prepared as described in the preceding paragraph (2.0 g, 5.0 millimoles-mmols), acetone (16 g), methacrylic anhydride (0.89 mL, 6.0 mmols), triethylamine (0.84 mL, 6.0 mmols), DMAP (30.5 mg, 0.25 mmols) and hydroquinone (20 mg) were added to a 50 mL round bottomed flask equipped with a magnetic stirrer, heating mantle and reflux condenser. The reaction mixture was stirred and heated to reflux for 50 minutes. A significant amount of insoluble material was present. DMF (10 mL) was added and the reaction mixture was stirred at reflux for about 10 minutes at which point a homogeneous solution was obtained. The reaction solution was stirred at reflux for an additional 1 h. The starting intermediate azo compound was consumed according to TLC analysis (1:1 THF/cyclohexane, R_{f} (intermediate azo compound)=0 to 0.55, R_{f} (product compound)=0.77). The reaction solution was allowed to cool to about 40° C. and a 1:1 solution of methanol in water (8 mL) was added at a dropwise rate to precipitate the product. The precipitate was collected by suction filtration and washed with 8 mL of a1:1 solution of methanol in water, then allowed to air dry on the filter overnight to give 1.08 g of yellow solid azo product (46 percent of the theoretical yield). The azo product has the following structure (supported by high performance liquid chromatography mass spectrometry—HPLC-MS): 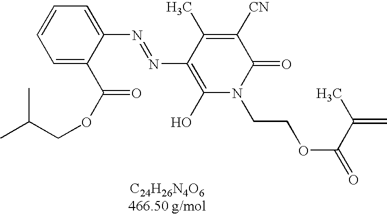
The azo product dissolved in DMF exhibited an absorption maximum at 427 nm (extinction coefficient=33,800) in the UV-visible light absorption spectrum.

### Example 3

Nitrosylsulfuric acid (40%, 6.4 g, approximately 0.02 mole) was added to a 100 mL round bottomed flask equipped with a magnetic stir bar and ice water bath. To the stirred solution was added 1:5 acid (20 mL) in small portions such that the temperature did not exceed 25° C. The reaction solution was further cooled and 2-ethylhexyl anthranilate (5.08 g, 0.02 mole) was added at about 0-5° C. with stirring and cooling, followed by an additional quantity (20 mL) of 1:5 acid. The diazotization reaction solution was stirred at 0-5° C. for about 2 hours, then added dropwise at less than about 5° C. to a solution of 3-cyano-6-hydroxy-1-(2′-hydroxyethyl)-4-methyl-2-pyridone potassium salt (4.64 g, 0.02 mole) dissolved in an ice water mixture (100 mL). The coupling mixture was allowed to sit in the ice water bath with occasional stirring about 45 minutes and diluted with water (100 mL). The yellow intermediate azo product was collected by vacuum filtration, washed hot water and air dried to give 8.45 g of yellow intermediate product (93% of the theoretical yield) having the following structure (confirmed by FDMS):

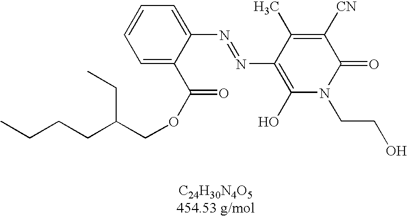

The intermediate azo compound prepared as described in the preceding paragraph (2.0 g, 4.4 mmols), acetone (16 g),methacrylic anhydride (0.79 mL, 5.3 mmols), triethylamine (0.74 mL, 5.3 mmols), DMAP (26.9 mg, 0.22 mmols) and hydroquinone (20 mg) were added to a 50 mL round bottomed flask equipped with a magnetic stirrer, heating mantle and reflux condenser. The reaction mixture was stirred and heated to reflux for 1 hour at which time the starting intermediate azo compound was consumed according to TLC analysis (1:1 THF/cyclohexane, R_{f} (intermediate compound)=0.55 to 0.68, R_{f} (azo product)=0.82). The reaction solution was allowed to cool to about 40° C. and a 1:1 solution of methanol in water (8 mL) was added at a dropwise rate to precipitate the product. The precipitate was collected by suction filtration and washed with 8 mL of a1:1 solution of methanol in water and then allowed to air dry on the filter overnight to give 1.33 g of yellow product (58 percent of the theoretical yield) having the following structure (confirmed by HPLC-MS): 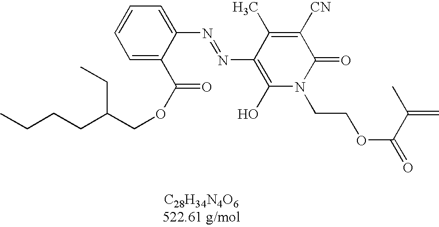
The product dissolved in DMF exhibited an absorption maximum at 423 nm (extinction coefficient=31,400) in the UV-visible light absorption spectrum.

### Example 4

Nitrosylsulfuric acid (40%, 6.4 g, approximately 0.02 mole) was added to a 100 mL round bottomed flask equipped with a magnetic stir bar and ice water bath. To the stirred solution was added 1:5 acid (20 mL) was added in small portions such that the temperature did not exceed 25° C. The reaction solution was further cooled and 2-(2′-ethoxyethyl) anthranilate (4.18 g, 0.02 mole) was added at about 0-5° C. with stirring and cooling, followed by an additional quantity (20 mL) of 1:5 acid. The diazotization reaction solution was stirred at 0-5° C. for about 2 hours then added dropwise at less than about 5° C. to a solution of 3-cyano-6-hydroxy-1-(2′-hydroxyethyl)-4-methyl-2-pyridone potassium salt (4.64 g, 0.02 mole) dissolved in an ice water mixture (100 mL). The coupling mixture was allowed to sit in the ice water bath with occasional stirring about 45 minutes and diluted with water (100 mL). The yellow azo intermediate product was collected by vacuum filtration, washed with hot water and air dried to give 6.3 g of yellow intermediate azo compound (76% of the theoretical yield) having the following structure (confirmed by FDMS):

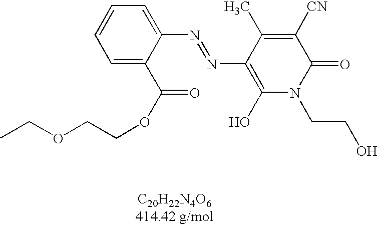

The intermediate azo compound prepared as described in the preceding paragraph (2.0 g, 4.8 mmols), acetone (16 g),methacrylic anhydride (0.86 mL, 5.8 mmols), triethylamine (0.81 mL, 5.8 mmols), DMAP (30.0 mg, 0.24 mmols) and hydroquinone (20 mg) were added to a 50 mL round bottomed flask equipped with a magnetic stirrer, heating mantle and reflux condenser. The reaction mixture was stirred and heated to reflux for 50 minutes. A significant amount of insoluble material was present. DMF (10 mL) was added and the reaction mixture was stirred at reflux for about 10 minutes at which point a homogeneous solution was obtained. The reaction solution was stirred at reflux for an additional 1 hour at which time the starting intermediate azo compound was consumed according to TLC analysis (1:1 THF/Cyclohexane, R_{f} (intermediate compound)=0 to 0.41, R_{f} (product compound)=0.71). The reaction solution was allowed to cool to about 40° C. and a 1:1 solution of methanol in water (8 mL) was added at a dropwise rate to precipitate the product. The precipitate was collected by suction filtration and washed with 8 mL of a1:1 solution of methanol in water then allowed to air dry on the filter overnight to give 1.27 g of yellow azo product (53 percent of the theoretical yield) having the structure (confirmed by HPLC-MS): 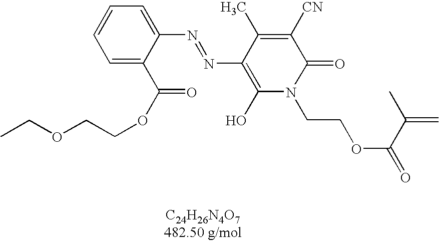
The azo product dissolved in DMF exhibited an absorption maximum at 427 nm (extinction coefficient=37,600) in the UV-visible light absorption spectrum.

### Examples 5-73

The azo compounds set forth in Table I conform to formula Ia and are further examples of the compounds provided by the present invention. These compounds may be prepared according to the procedures described above.

TABLE I

| | |
|---|---|
| | Ia |
| 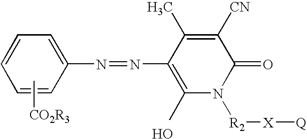 | |

| | | | R₃ (Position | |
|---|---|---|---|---|
| Example | X | R₂ | of —COOR₃) | Q |
| 5 | —O— | —(CH₂CH₂)— | —(CH₂)₂CH₃ (2) | —CONHC(CH₃)₂C₆H₄-3-C(CH₃)═CH₂ |
| 6 | —O— | —(CH₂CH₂)— | —CH(CH₃)₂ (2) | —CONHC(CH₃)₂C₆H₄-3-C(CH₃)═CH₂ |
| 7 | —O— | —(CH₂CH₂)— | —CH₂CH(CH₃)₂ (2) | —CONHC(CH₃)₂C₆H₄-3-C(CH₃)═CH₂ |
| 8 | —O— | —(CH₂CH₂)— | —CH(CH₃)₂ (2) | —COC(CH₃)═CH₂ |
| 9 | —O— | —(CH₂CH₂)— | —(CH₂)₂CH₃ (2) | —COC(CH₃)═CH₂ |
| 10 | —O— | —(CH₂CH₂)— | —(CH₂)₂OC₂H₅ (2) | —CONHC(CH₃)₂C₆H₄-3-C(CH₃)═CH₂ |
| 11 | —O— | —CH(CH₂OQ)CH₂— | —CH(CH₃)₂ (2) | —CONHCOC(CH₃)═CH₂ |
| 12 | —O— | —(CH₂CH₂)— | —(CH₂)₂CH₃ (4) | —CONHC(CH₃)₂C₆H₄-3-C(CH₃)═CH₂ |
| 13 | —O— | —(CH₂CH₂)— | —CH(CH₃)₂ (4) | —CONHC(CH₃)₂C₆H₄-3-C(CH₃)═CH₂ |
| 14 | —O— | —(CH₂CH₂)— | —CH₂CH(CH₃)₂ | —CONHC(CH₃)₂C₆H₄-3-C(CH₃)═CH₂ |
| 15 | —O— | —(CH₂CH₂)— | —CH(CH₃)₂ (4) | —COC(CH₃)═CH₂ |
| 16 | —O— | —(CH₂CH₂)— | —(CH₂)₂CH₃ (4) | —COC(CH₃)═CH₂ |
| 17 | —O— | —(CH₂CH₂)— | —(CH₂)₂OC₂H₅ (4) | —CONHC(CH₃)₂C₆H₄-3-C(CH₃)═CH₂ |
| 18 | —O— | —CH(CH₂OQ)CH₂— | —CH(CH₃)₂ (2) | —COC(CH₃)═CH₂ |
| 19 | —O— | —CH(CH₂OQ)CH₂— | —CH₂CH(C₂H₅)C₄H₉ (2) | —COC(CH₃)═CH₂ |
| 20 | —O— | —CH(CH₂OQ)CH₂— | —CH₂CH(C₂H₅)C₄H₉ (2) | —CONHC(CH₃)₂C₆H₄-3-C(CH₃)═CH₂ |
| 21 | —O— | —CH(CH₂OQ)CH₂— | —(CH₂)₂CH₃ (2) | —COC(CH₃)═CH₂ |
| 22 | —O— | —CH(CH₂OQ)CH₂— | —(CH₂)₂CH₃ (4) | —CONHC(CH₃)₂C₆H₄-3-C(CH₃)═CH₂ |
| 23 | —O— | —CH(CH₂OQ)CH₂— | —CH(CH₃)₂ (4) | —CONHC(CH₃)₂C₆H₄-3-C(CH₃)═CH₂ |
| 24 | —O— | —CH(CH₂OQ)CH₂— | —CH₂CH(CH₃)₂ (4) | —CONHC(CH₃)₂C₆H₄-3-C(CH₃)═CH₂ |
| 25 | —O— | —CH(CH₂OQ)CH₂— | —CH(CH₃)₂ (4) | —COC(CH₃)═CH₂ |
| 26 | —O— | —CH(CH₂OQ)CH₂— | —(CH₂)₂CH₃ (4) | —COC(CH₃)═CH₂ |
| 27 | —O— | —CH(CH₂OQ)CH₂— | —(CH₂)₂OC₂H₅ (4) | —CONHC(CH₃)₂C₆H₄-3-C(CH₃)═CH₂ |
| 28 | —O— | —CH₂CH₂OCH₂CH₂— | —CH₂CH(CH₃)₂ (2) | —CONHC(CH₃)₂C₆H₄-3-C(CH₃)═CH₂ |
| 29 | —O— | —CH₂CH₂CH₂— | —CH(CH₃)₂ (2) | —CONHC(CH₃)₂C₆H₄-3-C(CH₃)═CH₂ |
| 30 | —O— | —CH₂CH₂CH₂— | —CH₂CH(CH₃)₂ (2) | —CONHC(CH₃)₂C₆H₄-3-C(CH₃)═CH₂ |
| 31 | —O— | —CH₂CH₂CH₂— | —CH(CH₃)₂ (2) | —COC(CH₃)═CH₂ |
| 32 | —O— | —CH₂CH₂CH₂— | —(CH₂)₂CH₃ (2) | —COC(CH₃)═CH₂ |
| 33 | —O— | —CH₂CH₂CH₂— | —(CH₂)₂OC₂H₅ (2) | —CONHC(CH₃)₂C₆H₄-3-C(CH₃)═CH₂ |
| 34 | —O— | —CH₂CH₂CH₂CH₂— | —CH(CH₃)₂ (2) | —CONHC(CH₃)₂C₆H₄-3-C(CH₃)═CH₂ |
| 35 | —O— | —CH₂CH₂CH₂CH₂— | —CH₂CH(CH₃)₂ (2) | —CONHC(CH₃)₂C₆H₄-3-C(CH₃)═CH₂ |
| 36 | —O— | —CH₂CH₂CH₂CH₂— | —CH(CH₃)₂ (2) | —COC(CH₃)═CH₂ |
| 37 | —O— | —CH₂CH₂CH₂CH₂— | —(CH₂)₂CH₃ (2) | —COC(CH₃)═CH₂ |
| 38 | —O— | —CH₂CH₂CH₂CH₂— | —(CH₂)₂OC₂H₅ (2) | —CONHC(CH₃)₂C₆H₄-3-C(CH₃)═CH₂ |
| 39 | —O— | —(CH₂)₆— | —CH(CH₃)₂ (2) | —CONHC(CH₃)₂C₆H₄-3-C(CH₃)═CH₂ |
| 40 | —O— | —(CH₂)₆— | —CH₂CH(CH₃)₂ (2) | —CONHC(CH₃)₂C₆H₄-3-C(CH₃)═CH₂ |
| 41 | —O— | —(CH₂)₆— | —CH(CH₃)₂ (2) | —COC(CH₃)═CH₂ |
| 42 | —O— | —(CH₂)₆— | —(CH₂)₂CH₃ (2) | —COC(CH₃)═CH₂ |
| 43 | —O— | —(CH₂)₆— | —(CH₂)₂OC₂H₅ (2) | —CONHC(CH₃)₂C₆H₄-3-C(CH₃)═CH₂ |
| 44 | —O— | —(CH₂)₆— | —(CH₂)₂OC₂H₅ (2) | —CONHCOC(CH₃)═CH₂ |
| 45 | —O— | —CH₂CH₂OCH₂CH₂— | —(CH₂)₂OC₂H₅ (2) | —CONHC(CH₃)₂C₆H₄-3-C(CH₃)═CH₂ |
| 46 | —O— | —CH(CH₂OQ)CH₂— | —CH₂CH(CH₃)₂ (2) | —COC(CH₃)₂NHCOC(CH₃)═CH₂ |
| 47 | —O— | —CH(CH₂OQ)CH₂— | —(CH₂)₂OC₂H₅ (2) | —CONHCH₂CH₂OCOCC(CH₃)═CH₂ |
| 48 | —O— | —CH(CH₂OQ)CH₂— | —(CH₂)₂OC₂H₅ (2) | —COC(CH₃)₂NHCOC(CH₃)═CH₂ |
| 49 | —NH— | —CH(CH₂OQ)CH₂— | —(CH₂)₂OC₂H₅ (2) | —CONHC(CH₃)₂C₆H₄-3-C(CH₃)═CH₂ |
| 50 | —N(CH₃)— | —CH₂CH₂OCH₂CH₂— | —CH(CH₃)₂ (2) | —COC(CH₃)═CH₂ |
| 51 | —N(C₂H₅)— | —CH₂CH₂OCH₂CH₂— | —CH₂CH(C₂H₅)C₄H₉ (2) | —COC(CH₃)═CH₂ |
| 52 | —N(C₂H₅)— | —(CH₂CH₂— | —CH₂CH(C₂H₅)C₄H₉ (2) | —COCH═CHCO₂C₂H₅ |
| 53 | —N(C₂H₅)— | —(CH₂CH₂— | —CH₂CH(C₂H₅)C₄H₉ (2) | 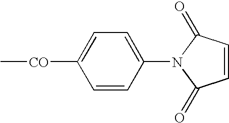 |
| 54 | —N(C₂H₅)— | —(CH₂CH₂— | —(CH₂CH₂O)₃C₂H₅ (4) | —CONHCOC(CH₃)═CH₂ |
| 55 | —N(C₂H₅)— | —CH₂CH₂OCH₂CH₂— | —(CH₂CH₂O)₃C₂H₅ (4) | —CONHC(CH₃)₂C₆H₄-3-C(CH₃)═CH₂ |
| 56 | —N(C₂H₅)— | —CH(CH₂OQ)CH₂— | —CH₂CH(CH₃)₂ (4) | —COC(CH₃)₂NHCOC(CH₃)═CH₂ |
| 57 | —N(CH₃)— | —CH(CH₂OQ)CH₂— | —(CH₂CH₂O)₂C₂H₅ (4) | —CONHCH₂CH₂OCOCC(CH₃)═CH₂ |
| 58 | —N(CH₃)— | —CH(CH₂OQ)CH₂— | —(CH₂CH₂O)₂C₂H₅ (4) | —COC(CH₃)₂NHCOC(CH₃)═CH₂ |
| 59 | —NH— | —CH(CH₂OQ)CH₂— | —(CH₂CH₂O)₂C₂H₅ (4) | —CONHC(CH₃)₂C₆H₄-3-C(CH₃)═CH₂ |
| 60 | —N(CH₃)— | —CH₂CH₂OCH₂CH₂— | —CH(CH₃)₂ (4) | —COC(CH₃)═CH₂ |
| 61 | —N(C₂H₅)— | —CH₂CH₂OCH₂CH₂— | —CH₂CH(C₂H₅)C₄H₉ (4) | —COC(CH₃)═CH₂ |
| 62 | —N(C₂H₅)— | —(CH₂CH₂— | —CH₂CH(C₂H₅)C₄H₉ (4) | —COCH═CHCO₂C₂H₅ |
| 63 | —N(C₂H₅)— | —(CH₂CH₂— | —CH₂CH(C₂H₅)C₄H₉ (4) | 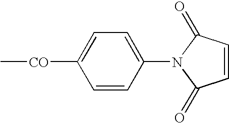 |
| 64 | —O— | —CH₂CH(CH₃)— | —CH(CH₃)CH₂CH₃ (2) | —CONHC(CH₃)₂C₆H₄-3-C(CH₃)═CH₂ |
| 65 | —O— | —CH₂CH(CH₃)— | —CH₂CH₂CH(CH₃)₂ (2) | —CONHC(CH₃)₂C₆H₄-3-C(CH₃)═CH₂ |
| 66 | —O— | —(CH₂CH₂)₂OCH₂CH₂— | —CH₂CH(C₂H₅)₂ (2) | —COC(CH₃)═CH₂ |
| 67 | —O— | —CH₂CH(CH₃)— | —CH₂C(CH₃)₃ (2) | —COC(CH₃)═CH₂ |
| 68 | —O— | —(CH₂)_{2—} | —C(CH₃)₂CH₂CH₃ (2) | —CONHC(CH₃)₂C₆H₄-3-C(CH₃)═CH₂ |
| 69 | —O— | —CH₂CH₂OCH₂CH₂— | —CH₂CH(CH₃)—(CH₂)₄CH₃ (2) | —CONHC(CH₃)₂C₆H₄-3-C(CH₃)═CH₂ |
| 70 | —O— | 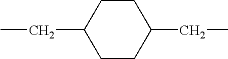 | —CH₂CH(C₂H₅)C₄H₉ (2) | —COC(CH₃)═CH₂ |
| 71 | —O— | —CH₂CH(CH₃)— | —CH₂CH(CH₃)—(CH₂)₂CH₃ (2) | —COC(CH₃)═CH₂ |
| 72 | —O— | —(CH₂)_{2—} | —CH₂CH(CH₃)—(CH₂)₂CH₃ (2) | —COC(CH₃)═CH₂ |
| 73 | —O— | —CH₂CH(CH₃)— | —CH₂CH(C₂H₅)₂ (2) | —COC(CH₃)═CH₂ |

### Comparative Example 1

Methyl 4-aminobenzoate (7.55 g, 0.05 mole) was added to 60% aqueous acetic acid (100 mL) and the solution stirred with cooling in an ice water bath. A solution of nitrosyl sulfuric acid was prepared by the gradual addition of sodium nitrite (3.6 g, 0.05 mole) to concentrated sulfuric acid (25 mL) with stirring and added gradually to the amine solution at about 5-10° C. with stirring and cooling. After about 1.5 hours, an aliquot (0.01 mole) of the diazonium salt solution was added to a solution of 3-cyano-N-(2′-hydroxyethyl )-6-hydroxy-4-methyl-2-pyridone (0.01 mole) dissolved in water (100 mL) containing sodium hydroxide (15.4 g of 50% aqueous NaOH solution) with ice present for cooling. The azo coupling mixture was allowed to stand for 1 hour with occasional stirring and diluted further with water. The yellow azo compound was collected by filtration, washed with water and dried in air to give 3.5 g of yellow intermediate azo compound having the structure set forth below. An absorption maximum was observed at 428 nm in an acetone solution in the UV-visible light absorption spectrum.

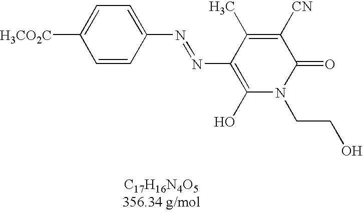

The intermediate azo compound (1.34 g, 0.00375 mole) prepared according to the procedure described in the preceding paragraph, 3-isopropenyl-α,α-dimethylbenzyl isocyanate (0.8 g, 0.00375 mole), toluene (40 mL) and dibutyltin dilaurate (3 drops) were mixed and heated with stirring at about 90° C. for 2.5 hours. TLC (1:1 tetrahydrofuran (THF)/cyclohexane) indicated some starting material (intermediate azo compound) remained. Additional 3-isopropenyl-α,α-dimethylbenzyl isocyanate (10 drops) was added and the reaction mixture stirred at about 90° C. for an additional hour to complete the reaction. The reaction mixture was allowed to cool and drowned into heptane (100 mL) to produce the solid yellow azo compound that was collected by filtration, washed with heptane and dried in air (yield—1.94 g, 93% of the theoretical yield). The azo compound product has the structure (supported by FD-MS) set forth below.

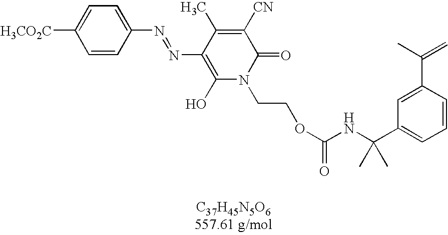

### Comparative Example 2

Sodium nitrite (3.6, 0.05 mole) was added portionwise with stirring to concentrated sulfuric acid (25.0 mL) allowing the temperature to rise. The clear yellow solution was cooled and 1:5 acid (50 mL) was added with stirring below about 20° C. After cooling the nitrosyl sulfuric acid solution, methyl anthranilate (7.56, 0.05 mole) was added at about 0-5° C. with stirring and cooling, followed by an additional quantity (50 mL) of 1:5 acid. The diazotization reaction solution was stirred at 0-5° C. for about 2 hours, then added dropwise at less than about 5° C. to a solution of 3-cyano-6-hydroxy-1-(2′-hydroxyethyl)-4-methyl-2-pyridone potassium salt (11.62 g, 0.05 mole, prepared in Example 1), dissolved in water (60 mL) that contained 50% sodium hydroxide (7.6 g, 0.095 mole). Ice was added as need to maintain the temperature below about 5° C. The coupling mixture was stirred for about 1.0 hour and diluted by the addition of water (400 mL), stirred for another hour and then treated with live steam to raise the temperature to about 80° C. The yellow intermediate azo compound was collected by vacuum filtration, washed with hot water and air dried to give 16.86 g of intermediate product (94.6% of the theoretical yield). Proton nuclear magnetic resonance spectroscopy (¹H NMR) confirmed the intermediate azo compound has the following structure:

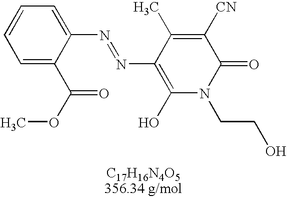

The intermediate azo compound (13.4 g, 0.0375 mole) prepared according to the procedure described in the preceding paragraph, 3-isopropenyl-α,α-dimethyl-benzyl isocyanate (8.0 g, 0.0375 mole), toluene (325 mL) and dibutyltin dilaurate (25 drops) were mixed and heated with stirring at 100° C.-120° C. for 1 hour. TLC (1:1 THF/cyclohexane) indicated some starting material remained. Additional 3-isopropenyl-α,α-dimethylbenzyl isocyanate (10 drops) was added and the reaction mixture stirred at about 90° C. for an additional hour to complete the reaction. The reaction mixture was allowed to cool and poured gradually into heptane (900 mL) to produce the yellow solid product that was collected by filtration. The filter cake was reslurried in a mixture of heptane (400 mL) and toluene (100 mL) and the product was collected by filtration, washed with heptane and dried in air (yield—20.4 g, 94% of the theoretical yield). The azo product has the structure (supported by FD-MS and ¹H NMR) set forth below. 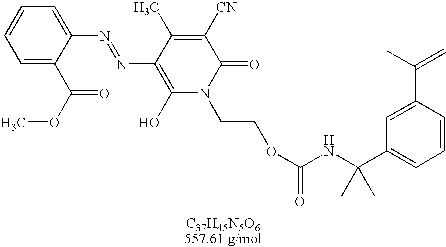
An absorption maximum at 428 nm (extinction ceofficient=35,300) was observed in the UV-visible light absorption spectrum of the azo product dissolved in DMF.

### Comparative Example 3

The intermediate azo compound prepared in Comparative Example 2 (2.0 g, 5.6 mmols), acetone (16 g), methacrylic anhydride (1.0 mL, 6.7 mmols), triethylamine (0.93 mL, 6.7 mmols), 4-(dimethylamino)pyridine (DMAP, 34.2 mg, 0.28 mmols) and hydroquinone (20 mg) were added to a 50 mL round bottomed flask equipped with a magnetic stirrer, heating mantle and reflux condenser. The reaction mixture was stirred and heated to reflux for 50 minutes. A significant amount of insoluble material was present. DMF (10 mL) was added and the reaction mixture was stirred at reflux for about 10 minutes. A significant amount of insoluble material was present. Another 10 mL of DMF was added at which point a homogeneous solution was obtained. The reaction solution was stirred at reflux for an additional 2 h. The intermediate azo compound reactant was consumed according to TLC analysis (1:1 THF/cyclohexane, R_{f} (intermediate azo compound)=0 to 0.41, R_{f} (azo product compound)=0.71). The reaction solution was allowed to cool to room temperature and stir for about 2 hours. A 1:1 solution of methanol in water (8 mL) was added at a dropwise rate to precipitate the product. The precipitate was collected by suction filtration and washed with 8 mL of a 1:1 solution of methanol in water then allowed to air dry on the filter overnight to give 1.15 g of a solid yellow azo compound product (48 percent of the theoretical yield). The product has the following structure that is supported by high performance liquid chromatography mass spectrometry—HPLC-MS: 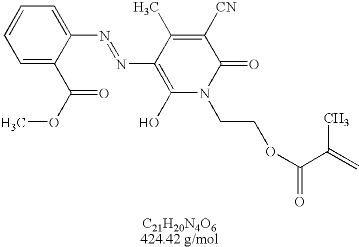
An absorption maximum at 428 nm (extinction ceofficient=34,300) was observed in the UV-visible light absorption spectrum of the dye dissolved in DMF.

### Comparative Example 4

Methyl cyanoacetate (198 g, 2.0 moles) was added to a 2 L, 4-neck flask equipped with a condenser, a Dean-Stark trap, heating mantle and thermocouple. The methyl cyanoacetate was stirred under nitrogen and heated to about 95° C. and 3-amino-1-propanol (122 g, 2.0 moles) was added dropwise over 2 hours while methanol was collected in a Dean-Stark trap. The reaction mixture was heated to about 150° C. and held at that temperature until methanol distillation stopped (about 1 hour). The reaction mixture was allowed to cool to room temperature. Ethyl acetoacetate (260 g, 2.0 moles) and methanol (250 mL) were added and the reaction mixture was heated to reflux. A solution of potassium hydroxide (125 g) dissolved in. methanol (300 mL) was added dropwise and refluxing was continued for about 6 hours. The reaction mixture was allowed to cool and a snail amount of white solid was collected by filtration. The white solid was determined not to be the product. The mother liquor was acidified by adding 130 mL of concentrated HCl in small portions, which precipitated the product. The reaction mixture was cooled to 10 to 15° C. and the product was collected by vacuum filtration. The white solid was washed thoroughly with cold water and allowed to air dry overnight to give 161.5 g of pyridone product (33% of theory) having the following structure (confirmed by FDMS):

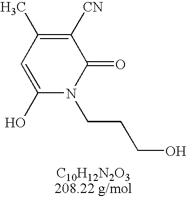

Water (20 mL)and 4-vinylaniline (2.38 g, 0.02 mole) were mixed with concentrated HCl (6 mL) in a 100 mL flask cooled using an ice water bath and added in small portions to a solution of sodium nitrite (1.44 g) in water (5 mL) such that the reaction temperature did not exceed 5° C. The diazotization reaction solution was stirred at 0-5° C. for about 2 hours and then added dropwise at less than about 5° C. to a solution of 3-cyano-6-hydroxy-1-(3′-hydroxypropyl)-4-methyl-2-pyridone potassium salt (4.16 g, 0.02 mole, prepared as described in the preceding paragraph) and potassium hydroxide (8.0 g) that was dissolved in an ice water mixture(125 mL). The coupling mixture was made acidic by adding acetic acid. The reaction was allowed to proceed for 1 hour at 0-5° C., then drowned into water to precipitate the product. The yellow azo product was collected by vacuum filtration, washed with hot water and air dried to give 3.14 g of intermediate azo product (46% of the theoretical yield) having the structure (confirmed by FDMS):

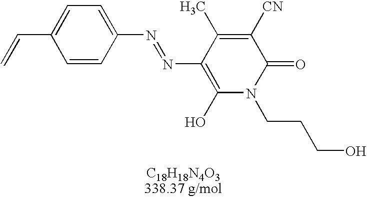

The intermediate azo compound prepared in the preceding paragraph (1.0 g, 2.96 mmols), acetone (10 g), acryloyl chloride (0.26 mL, 3.25 mmols), triethylamine (0.45 mL, 3.25 mmols), DMAP (18.0 mg, 0.15 mmols) and hydroquinone (10 mg) were added to a 50 mL round bottomed flask equipped with a magnetic stirrer, heating mantle and reflux condenser. The reaction mixture was heated to reflux for 20 minutes and some insoluble material was present. DMF (10 mL) was added and the reaction mixture was heated to reflux. It appeared that a homogeneous solution was obtained. The reaction mixture was heated for about 30 minutes and TLC analysis revealed that some starting material remained. The reaction mixture was allowed to cool to room temperature and another 0.26 mL of acryloyl chloride was added. The reaction mixture was returned to reflux. The reaction mixture appeared very dark and contained some large chunks or pieces of insoluble material. The reaction mixture was allowed to cool to room temperature and 20 mL of a 50% solution of methanol in water was added at a dropwise rate to precipitate the product. Some of the large black chucks were removed and found to be insoluble in DMF and acetone. It was believed that the desired azo product (Example 107 of U.S. Pat. No. 5,188,641) had polymerized before completion of the reaction.

The solubilites of the reactive azo compounds of Examples 1, 2, 3 and 4 and Comparative Examples 1, 2 and 3 (C-1, C-2 and C-3) in toluene were determined according to the following procedures: A sample (0.2 g) of each compound was added to a screw top vial followed by the addition of toluene (0.5 mL for each vial containing the reactive azo compound of Examples 1, 3, 4 or C-3; 1.0 mL for Examples 2 or C-1; 1.5 mL for Example C-2. Each vial was capped and heated until the solution or mixture began to boil. Each solution or mixture then was allowed to cool to room temperature. Various amounts of toluene were required to give a filterable solution or suspension upon cooling. The solutions or suspensions of the azo compounds were filtered (0.45 μm nylon membrane with glass microfiber prefilter, Autovial®, Whatman Inc.) to give homogenous solutions (filtrates). Each filtered solution was weighed and the toluene solvent was removed under reduced pressure to give the mass of dry residue of each azo compound dissolved in the toluene solvent. The weight percent solubility of each azo compound is defined as

$$WPS = \frac{Mass of Dry Azo Compound Residue}{Mass of Toluene Solution Filtrate} \times 100$$

wherein WPS is the weight percent solubility for each azo compound in toluene and Dry Azo Compound Residue and Toluene Solution Filtrate are produced as described above. The results of the toluene solubility evaluations are shown in Table II wherein Azo compound refers to the example in which the reactive azo compound and its preparation is described.

TABLE II

| Azo Compound | Mass of Toluene | Mass of Dry Azo | |
|---|---|---|---|
| of Example | Solution Filtrate | Compound Residue | WPS |
| 1 | 0.4 g | 0.15 g | 37.5 |
| 2 | 0.86 g | 0.16 g | 18.6 |
| 3 | 0.15 g | 0.05 g | 33.3 |
| 4 | 0.31 g | 0.11 g | 35.5 |
| C-1 | 0.57 g | 0.05 g | 8.8 |
| C-2 | 0.88 g | 0.02 g | 2.3 |
| C-3 | 0.09 g | 0.01 g | 11.1 |

The data presented in Table II demonstrate the improved solubility possessed by the polymerizable azo compounds of the present invention relative to compounds of similar structure. Comparative Example 3 differs from Example 2, Example 3 and Example 4 only by the alkyl ester substituents. The methyl ester derivative has much lower solubility in toluene than do the yellow azo compounds of the present invention. The solubility of Comparative Examples C-1, C-2 and C-3 was much lower than the yellow azo dyes of the present invention regardless of the position of the methyl ester substituent on the diazotizable aromatic amine. The yellow azo compounds of the present invention can be prepared as concentrated solutions and diluted with other solvents or ethylenically-unsaturated, free radically polymerizable monomers as needed to obtain the appropriate color strength.

The coating compositions provided by the present invention comprise at least one of the azo colorant compounds of formula I and at least one polymerizable, ethylenically-unsaturated composition. The azo colorant compounds of formula I containing one or more ethylenically-unsaturated, or vinyl including substituted vinyl, groups, may be polymerized with the ethylenically-unsaturated composition to provide yellow polymeric film-forming materials useful as coatings on a variety of substrates. The polymerization may be accomplished by conventional addition polymerization procedures involving free radical mechanisms, e.g., wherein free radicals are generated by exposure to ultraviolet (UV) light by methods known in the art of preparing UV-cured resins. Polymerization may be facilitated by the addition of one or more photoinitiators. The colored coating compositions typically are prepared by dissolving one or more azo colorant compounds containing copolymerizable groups in one or more polymerizable, ethylenically-unsaturated (or vinyl) compositions.

The polymerizable, ethylenically-unsaturated compositions useful in the coating compositions of the present invention are colorless or substantially colorless compounds and/or compositions capable of undergoing addition polymerization, e.g., upon exposure to UV light or radiation in the presence of a photoinitiator. A preferred embodiment of the coating compositions comprises radiation-curable compositions. Examples of such ethylenically-unsaturated compounds include monomer compounds such as acrylic acid, methacrylic acid and their anhydrides; crotonic acid; itaconic acid and its anhydride; cyanoacrylic acid and its esters; esters of acrylic and methacrylic acids such as allyl, methyl, ethyl, n-propyl, isopropyl, butyl, tetrahydrofurfuryl, cyclohexyl, isobornyl, n-hexyl, n-octyl, isooctyl, 2-ethylhexyl, lauryl, stearyl, and benzyl acrylate and methacrylate; and diacrylate and dimethacrylate esters of ethylene and propylene glycols, 1,3-butylene glycol, 1,4-butanediol, diethylene and dipropylene glycols, triethylene and tripropylene glycols, 1,6-hexane-diol, neopentyl glycol, polyethylene glycol, and polypropylene glycol, ethoxylated bisphenol A, ethoxylated and propoxylated neopentyl glycol; triacrylate and trimethacrylate esters of tris-(2-hydroxyethyl)isocyanurate, trimethylolpropane, ethoxylated and propoxylated trimethylolpropane, pentaerythritol, glycerol, ethoxylated and propoxylated glycerol; tetraacrylate and tetramethacrylate esters of pentaerythritol and ethoxylated and propoxylated pentaerythritol; acrylonitrile; vinyl acetate; vinyl toluene; styrene; N-vinyl pyrrolidinone; alpha-methylstyrene; maleate/fumarate esters; maleic/fumaric acid; crotonate esters, and crotonic acid.

The polymerizable, ethylenically-unsaturated compositions useful in the coating compositions of the present invention include polymers that contain ethylenically-unsaturated groups capable of undergoing polymerization upon exposure to UV radiation in the presence of a photoinitiator. The preparation and application of these polymerizable, ethylenically-unsaturated, polymeric compositions are described in the literature, e.g., Chemistry and Technology of UV and EB Formulation for Coatings, Inks, and Paints, Volume II: Prepolymers and Reactive Diluents, G. Webster, editor, John Wiley and Sons, London, 1997. Examples of such polymeric, polymerizable compositions include acrylated and methacrylated polyesters, acrylated and methacrylated polyethers, acrylated and methacrylated epoxy polymers, acrylated or methacrylated urethanes, acrylated or methacrylated polyacrylates (polymethacrylates), and unsaturated polyesters. The acrylated or methacrylated polymers and oligomers typically are combined with monomers which contain one or more acrylate or methacrylate groups, e.g., monomeric acrylate and methacrylate esters, and serve as reactive diluents. The unsaturated polyesters, which are prepared by standard polycondensation techniques known in the art, are most often combined with either styrene or other monomers, which contain one or more acrylate or methacrylate groups and serve as reactive diluents. The coating compositions of the present invention may comprise at least one of the azo colorant compounds of formula I and a combination of an unsaturated polyester and at least one monomer compound that contains two or more vinyl ether groups or two or more vinyl ester groups, e,g, as described in WO 96/01283, WO 97/48744, and EP 0 322 808).

The coating compositions of the present invention optionally may contain one or more added inert organic solvents if desired to facilitate application and coating of the compositions onto a substrate. Typical examples of suitable solvents include ketones, alcohols, esters, chlorinated hydrocarbons, glycol ethers, glycol esters, and mixtures thereof. Specific examples include, but are not limited to acetone, 2-butanone, 2-pentanone, ethyl acetate, propyl acetate, isopropyl acetate, butyl acetate, isobutyl acetate, ethylene glycol diacetate, ethyl 3-ethoxypropionate, methanol, ethanol, propanol, isopropanol, butanol, ethylene glycol, propylene glycol, ethylene glycol monomethyl ether, ethylene glycol monoethyl ether, ethylene glycol monopropyl ether, ethylene glycol monobutyl glycol, diethylene glycol monomethyl ether, diethylene glycol monoethyl ether, diethylene glycol monobutyl ether, propylene glycol monomethyl ether, diethylene glycol monobutyl ether acetate, diethylene glycol monoethyl ether acetate, ethylene glycol monobutyl ether acetate, propylene glycol monomethyl ether acetate, methylene chloride, chloroform, and mixtures thereof. The amount of added or extraneous solvent which may be present in our novel coating compositions may be in the range of about 1 to about 40 weight percent, more typically about 1 to about 25 weight percent, based on the total weight of the coating composition.

Certain polymerizable, ethylenically-unsaturated (vinyl) monomer compounds may serve as both reactant and solvent. These contain at least one unsaturated group capable of undergoing polymerization upon exposure to UV radiation in the presence of a photoinitiator. Specific examples include, but are not limited to: methacrylic acid, acrylic acid, ethyl acrylate and methacrylate, methyl acrylate and methacrylate, hydroxyethyl acrylate and methacrylate, diethylene glycol diacrylate, trimethylolpropane triacrylate, 1,6hexanediol di(meth)acrylate, neopentyl glycol diacrylate and methacrylate, vinyl ethers, divinyl ethers such as diethyleneglycol divinyl ether, 1,6-hexanediol divinyl ether, cyclohexanedimethanol divinyl ether, 1,4-butanediol divinyl ether, triethyleneglycol divinyl ether, trimethylolpropane divinyl ether, and neopentyl glycol divinyl ether, vinyl esters, divinyl esters such as divinyl adipate, divinyl succinate, divinyl glutarate, divinyl 1,4-cyclohexanedicarboxylate, divinyl 1,3-cyclohexanedicarboxylate, divinyl isophthalate, and divinyl terephthalate, N-vinyl pyrrolidone, and mixtures thereof.

The azo colorant compounds of formula I may be dispersed in water rather than dissolved in a solvent to facilitate application and coating of the substrate surface. In the water-dispersed compositions of the present invention a co-solvent may be optionally used. Typical examples of suitable cosolvents include but are not limited to acetone, 2-butanone, methanol, ethanol, isopropanol, ethylene glycol monomethyl ether, ethylene glycol monoethyl ether, ethylene glycol monopropyl ether, and ethylene glycol monobutyl ether, ethylene glycol, and propylene glycol. Typical examples of water-soluble ethylenically unsaturated solvents include but are not limited to: methacrylic acid, acrylic acid, N-vinyl pyrrolidone, 2-ethoxyethyl acrylate and methacrylate, polyethylene glycol dimethacrylate, polypropylene glycol monoacrylate and monomethacrylate, and mixtures thereof. The amount of suitable aqueous organic solvent (i.e., organic solvent and water) in the dispersed coating compositions of the present invention is about 10 to about 90 wt %, preferably about 75 to about 90 weight percent of the total coating composition.

The concentration of the azo compound or compounds of formula I in the coating compositions may be from about 0.005 to 30.0, preferably from about 0.5 to 25, weight percent based on the weight of the polymerizable vinyl compound(s) present in the coating composition. The coating compositions normally contain a photoinitiator. The amount of photoinitiator typically is about 1 to 15 weight percent, preferably about 3 to 5 weight percent, based on the weight of the polymerizable vinyl compound(s) present in the coating composition. Typical photoinitiators include benzoin and benzoin ethers such as marketed under the tradenames ESACURE BO, EB1, EB3, and EB4 from Fratelli Lamberti; VICURE 10 and 30 from Stauffer; benzil ketals such as 2,2-dimethoxy-1,2-diphenylethan-1-one (IRGACURE 651), 2-hydroxy-2-methyl-1-phenylpropan-1-one (IRGACURE 1173), 2-methyl-2-morpholino-1-(p-methylthiophenyl)propan-1-one (IRGACURE 907), alpha-hydroxyalkyl-phenones such as (1-hydroxycyclohexyl)(phenyl)-methanone (IRGACURE 184), 2-benzyl-2-(dimethylamino)-1-(4-morpholino-phenyl)butan-1-one (IRGACURE 369), 2-hydroxy-2-methyl-1-phenylpropan-1-one IRGACURE 1173) from Ciba Geigy, Uvatone 8302 by Upjohn; alpha, alpha-dialkoxyacetophenone derivatives such as DEAP and UVATONE 8301 from Upjohn; DAROCUR 116, 1173, and 2959 by Merck; and mixtures of benzophenone and tertiary amines In pigmented coating compositions, the rate of cure can be improved by the addition of a variety of phosphine oxide photoinitiaters such as bis(2,4,6-trimethylbenzoyl)-phenyl-phosphine oxide (Irganox 819), Irgacure 819, 1700, and 1700 and phosphine oxide mixtures such as a 50/50 by weight mixtures of IRGACURE 1173 and 2,4,6-trimethylbenzoyldiphenylphosphine oxide (DAROCUR 4265) from Ciba. Further details regarding such photoinitiators and curing procedures may be found in the published literature such as U.S. Pat. No. 5,109,097, incorporated herein by reference. Depending upon the thickness of the coating (film), product formulation, photoinitiator type, radiation flux, and source of radiation, exposure times to ultraviolet radiation of about 0.5 second to about 30 minutes (50-5000 mJ/square cm) typically are required for curing. Curing also can occur from solar radiation, i.e., sunshine.

The coating compositions of the present invention may contain one or more additional components typically present in coating compositions. Examples of such additional components include leveling, rheology, and flow control agents such as silicones, fluorocarbons or cellulosics; flatting agents; pigment wetting and dispersing agents; surfactants; ultraviolet (UV) absorbers; UV light stabilizers; tinting pigments; defoaming and antifoaming agents; anti-settling, anti-sag and bodying agents; anti-skinning agents; anti-flooding and anti-floating agents; fungicides and mildewcides; corrosion inhibitors; plasticizers; thickening agents; and/or coalescing agents. The coating compositions of the present invention also may contain non-reactive modifying resins. Typical non-reactive modifying resins include homopolymers and copolymers of acrylic and methacrylic acid; homopolymers and copolymers of alkyl esters of acrylic and methacrylic acid such as methyl, ethyl, n-propyl, isopropyl, butyl, tetrahydrofurfuryl, cyclohexyl, isobornyl, n-hexyl, n-octyl, isooctyl, 2-ethylhexyl, lauryl, stearyl, and benzyl acrylate and methacrylate; acrylated and methacrylated urethane, epoxy, and polyester resins, silicone acrylates, cellulose esters such as cellulose acetate butyrates, cellulose acetate, propionates, nitro-cellulose, cellulose ethers such as methyl cellulose, ethyl cellulose, hydroxypropyl cellulose, and hydroxypropyl methyl cellulose. Typical plasticizers include alkyl esters of phthalic acid such as dimethyl phthalate, diethyl phthalate, dipropyl phthalate, dibutyl phthalate, and dioctyl phthalate; citrate esters such as triethyl citrate and tributyl citrate; triacetin and tripropionin; and glycerol monoesters such as Eastman 18-04, 18-07, 18-92 and 18-99 from Eastman Chemical Company. Specific examples of additional additives can be found in Raw Materials Index, published by the National Paint & Coatings Association, 1500 Rhode Island Avenue, N.W., Washington, D.C. 20005.

The polymeric coatings produced from the coating compositions of the present invention typically have a solvent resistance of at least 100 MEK double rubs using ASTM Procedure D-3732; preferably a solvent resistance of at least about 200 double rubs. Such coatings also typically have a pencil hardness of greater than or equal to F using ASTM Procedure D-3363; preferably a pencil hardness of greater than or equal to H. The coating compositions can be applied to substrates with conventional coating equipment. The coated substrates are then exposed to radiation such as ultraviolet light in air or in nitrogen which gives a cured finish. Mercury vapor or Xenon lamps are applicable for the curing process. The coatings of the present invention can also be cured by electron beam.

Radiation-curable coating compositions are suitable as adhesives and coatings for such substrates as metals such as aluminum and steel, plastics, glass, wood, paper, and leather. On wood substrates the coating compositions may provide both overall transparent color and grain definition. Various aesthetically-appealing effects can be achieved thereby. Due to reduced grain raising and higher film thicknesses, the number of necessary sanding steps in producing a finished wood coating may be reduced when using the colored coating compositions of the invention rather than conventional stains. Coating compositions within the scope of our invention may be applied to automotive base coats where they can provide various aesthetically-appealing effects in combination with the base coats and color differences dependent on viewing angle (lower angles create longer path lengths and thus higher observed color intensities). This may provide similar styling effects as currently are achieved with metal flake orientation in base coats. Coating compositions within the scope of our invention may be applied to window films that may be suitable for automotive and architectural applications. Coating compositions within the scope of our invention may be applied to glass such as a fiber optic cable.

The present invention includes a substrate coated with a coating comprising a polymeric material derived from an ethylenically-unsaturated composition having copolymerized therein at least one of the azo compounds of formula I. The coated substrate may be a polymeric material, e.g., a film, sheeting or shaped article of a thermoplastic polymer, glass, wood, metal, paper and the like. Polyesters, acrylics and polycarbonate a preferred thermoplastic polymers.

The invention has been described in detail with particular reference to preferred embodiments thereof, but it will be understood that variations and modifications can be effected within the spirit and scope of the invention.

We claim:

1. An azo compound having the formula: 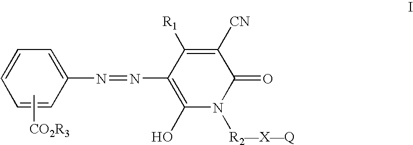
wherein:
R₁ is C₁-C₆-alkyl or aryl;
R₂ is C₂-C₁₀-alkylene, —CH₂-cyclohexylene-CH₂— or —(C₂-C₄-alkylene-Y)ₙ-C₂-C₄-alkylene;
R₃ is C₃-C₁₂-alkyl, C₃-C₈-cycloalkyl or —(C₂-C₄-alkylene-Y)ₙ—R₄;
R₄ is hydrogen, C₁-C₆-alkyl, substituted-C₁-C₆-alkyl, C₃-C₈-cycloalkyl or aryl;
X is —O—, —NH—, —N(SO₂R₅)— or —N(R₅)—;
R₅ is C₁-C₆-alkyl, C₃-C₈-cycloalkyl or aryl;
Y is —O—, —S—, —N(R₅)—, —N(COR₅)— or —N(SO₂R₅)—;
n is 1 to 3; and
Q is at least one ethylenically unsaturated, polymerizable group selected from the group consisting of: 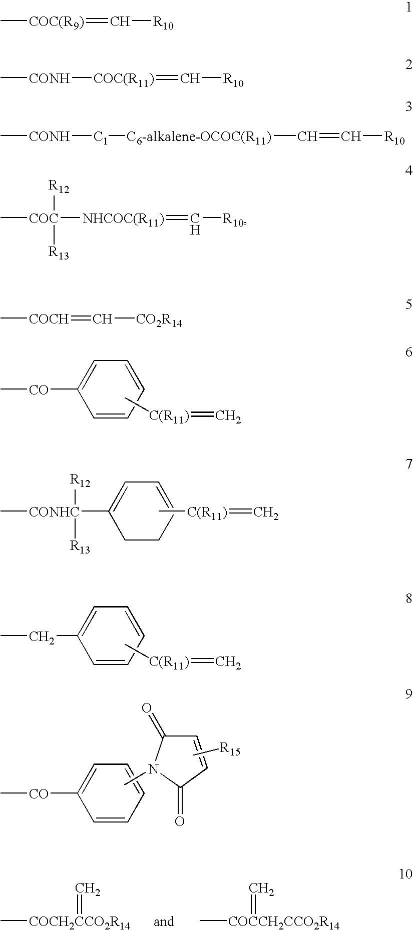
wherein:
R₉ is C₁-C₆-alkyl;
R₁₀ is hydrogen; C₁-C₆-alkyl; phenyl; phenyl substituted with one or more groups selected from the group consisting of C₁-C₆-alkyl, C₁-C₆-alkoxy, —N(C₁-C₆-alkyl), nitro, cyano, C₁-C₆-alkoxycarbonyl, C₁-C₆-alkanoyloxy and halogen; 1- or 2-naphthyl; 1- or 2-naphthyl substituted with C₁-C₆-alkyl or C₁-C₆-alkoxy; 2- or 3-thienyl; 2- or 3-thienyl substituted with C₁-C₆-alkyl or halogen; 2- or 3-furyl; or 2- or 3-furyl substituted with C₁-C₆-alkyl;
R₁₁ is hydrogen or C₁-C₆-alkyl;
R₁₂ and R₁₃ are, independently, hydrogen, C₁-C₆-alkyl, or aryl; or R₁₂ and R₁₃ may be combined to represent a —[—CH₂—]₃₋₅— radical;
R₁₄ is hydrogen, C₁-C₆-alkyl, C₃-C₈-alkenyl, C₃-C₈-cycloalkyl or aryl; and
R₁₅ is hydrogen, C₁-C₆-alkyl or aryl.

2. The azo compound according to claim 1 wherein Q is an ethylenically unsaturated, polymerizable group having the formula:
—COC(R₉)═CH—R₁₀ or 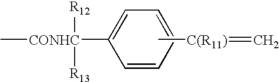
wherein R₁₀ is hydrogen and R₉, R₁₁, R₁₂ and R₁₃ are each methyl.

3. The azo compound according to claim 1 wherein —CO₂—R₃ is bonded at the 2-position relative to the azo group and R₃ is branched alkyl groups containing from four to eight carbon atoms or —CH₂CH₂(OCH₂CH₂)₁₋₃O—R₄, wherein R₄ is C₁-C₆-alkyl; R₁ is methyl; and Q is an ethylenically unsaturated, polymerizable group having the formula:
—COC(R₉)═CH—R₁₀ or 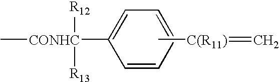
wherein R₁₀ is hydrogen and R₉, R₁₁, R₁₂ and R₁₃ are each methyl.

4. A coating composition comprising (i) at least one polymerizable vinyl compound, (ii) at least one azo compound according to claim 1, and (iii) a photoinitiator.

5. A coating composition comprising (i) at least one polymerizable vinyl compound, (ii) at least one azo compounds according to claim 2 present in a concentration of about 0.05 to 15 weight percent based on the weight of component (i), and (iii) a photoinitiator present in a concentration of about 1 to 15 weight percent based on the weight of the polymerizable vinyl compound(s) present in the coating composition.

6. The coating composition according to claim 5, wherein the polymerizable vinyl compound comprises a solution of a polymeric, polymerizable vinyl compound selected from the group consisting of acrylated polyester, methacrylated polyester, acrylated polyether, methacrylated polyether, acrylated epoxy polymer, methacrylated epoxy polymer, acrylated urethane, methacrylated urethane, and mixtures thereof, in a diluent selected from monomeric acrylate and methacrylate esters.

7. A coating composition comprising a polymer of one or more acrylic acid esters, one or more methacrylic acid esters and/or other co-polymerizable vinyl compounds, having copolymerized therein one or more of the azo compounds according to claim 1.

8. A composition comprising an acrylic polymer of one or more acrylic acid esters, one or more methacrylic acid esters or a mixture thereof having copolymerized therein one or more of the azo compounds according to claim 2.

9. A composition comprising an unsaturated polyester containing one or more maleate/fumarate residues; one or more monomers which contain one or more vinyl ether groups, one or more vinyl ester groups, or a combination thereof, and, optionally, one or more acrylic or methacrylic acid esters; or a mixture thereof having copolymerized therein one or more of the azo compounds defined in claim 2.

10. The composition according to claim 8, wherein said azo compound is present in an amount from about 0.05 to 15.0 weight percent based on the weight of the coating.

11. A coating resulting from the copolymerization of at least one azo compound according to claim 1 with one or more ethylenically-unsaturated monomers.

12. A coating resulting from the copolymerization of the coating composition according to claim 6.
